(19) United States
(12) Patent Application Publication
(10) Pub. No.: US 2025/0375170 A1
(43) Pub. Date: Dec. 11, 2025

(54) MODULE FOR COMPUTED TOMOGRAPHY DEVICE, AND COMPUTED TOMOGRAPHY DEVICE

(71) Applicant: SSTLabs Co., Ltd., Seongnam-si (KR)

(72) Inventors: Kwang Yun CHOI, Gwangju-si (KR); Chun He ZHENG, Gwangju-si (KR); Hyeong Kyu PARK, Seongnam-si (KR); Cheol Soo SONE, Seoul (KR)

(73) Assignee: SSTLabs Co., Ltd., Seongnam-si (KR)

(21) Appl. No.: 19/241,094

(22) Filed: Jun. 17, 2025

Related U.S. Application Data
(63) Continuation of application No. PCT/KR2023/021976, filed on Dec. 29, 2023.

(30) Foreign Application Priority Data
Dec. 29, 2022 (KR) 10-2022-0189140
Dec. 28, 2023 (KR) 10-2023-0194570

(51) Int. Cl. A61B 6/03; A61B 6/00; A61B 6/06; G21F 1/08

(52) U.S. Cl. CPC A61B 6/035; A61B 6/06; A61B 6/4411; G21F 1/085

(57) ABSTRACT

Provided are a computed tomography (CT) device and modules for the CT device. The module includes a gantry for providing an internal space through which a subject is transported, and having a first surface corresponding to an outer circumferential surface and a second surface corresponding to an inner circumferential surface, a source disposed on the second surface of the gantry to generate and emit radiation toward the subject, and a detector disposed opposite the source on the second surface of the gantry to detect the radiation transmitted through the subject.

### CROSS-REFERENCE TO RELATED APPLICATIONS

This application is a continuation of International Application No. PCT/KR2023/021976 filed on Dec. 29, 2023, which claims priority to Korean Patent Application No. 10-2022-0189140 filed on Dec. 29, 2022 and Korean Patent Application No. 10-2023-0194570 filed on Dec. 28, 2023, the entire contents of which are herein incorporated by reference.

### TECHNICAL FIELD

The present invention relates to a computed tomography (CT) device and modules for the CT device. Specifically, the present invention relates to a CT device using X-rays, and more particularly, to a CT device capable of implementing a stationary gantry type through a module assembly structure, and modules for the CT device.

### BACKGROUND ART

X-ray computed tomography (CT) is used in various clinical fields, e.g., diagnosis, real-time imaging during surgery, and postoperative prognosis evaluation. In addition, CT is applied not only in medical imaging devices but also for purposes such as airport cargo inspection and nondestructive inspection of industrial products like microstructures.

A CT device irradiates a subject with X-rays, some of which are absorbed by the subject and the others of which are detected by a plurality of detectors arranged in a linear or planar shape. Then, output data from the detectors are converted into electrical signals to reconstruct an image, thereby acquiring a tomographic image of the subject.

According to Korean Patent Application No. 1994-0028999 and others, a conventional CT device, which rotates a gantry installed around a subject to acquire X-ray transmission data, has difficulties with power supply to the gantry, real-time transmission of large data volumes, and precise positional control. Furthermore, CT requires a long time to acquire tomographic image data, and reconstructing the acquired transmission data into a 3D image also takes a considerable amount of time, making real-time use of CT during surgery technically difficult.

Conventionally, a CT device in which multiple sources are used and arranged with detectors in four pairs to cover approximately 90° per axis has been disclosed. Although the above device has a relatively simple structure, the detectors corresponding to the multiple sources are high-priced. Moreover, scanning takes a long time because X-ray beams are emitted sequentially from the multiple sources, and high-resolution imaging is hindered by signal distortion and scatter between the multiple sources.

### DETAILED DESCRIPTION OF THE INVENTION

### Technical Problem

The present invention provides a computed tomography (CT) device capable of freely assembling a plurality of modules to minimize the effect of scatter noise between fan beams, and modules for the CT device.

The present invention also provides a CT device capable of suitably combining modules in consideration of factors such as the purpose of use of the device, scanning speed, image resolution, and subject size, and modules for the CT device.

The present invention also provides a CT device capable of conveniently maintaining the entire device by replacing or repairing only a specific module, and modules for the CT device.

The present invention also provides a CT device capable of disposing a source and detector in a gantry to minimize external radiation leakage, and of improving shielding efficiency between modules, and modules for the CT device.

However, the above description is an example, and the scope of the present invention is not limited thereto.

### Technical Solution

According to an aspect of the present invention, there are provided modules for a computed tomography (CT) device, the modules including a gantry for providing an internal space through which a subject is transported, and having a first surface corresponding to an outer circumferential surface and a second surface corresponding to an inner circumferential surface, a source disposed on the second surface of the gantry to generate and emit radiation toward the subject, and a detector disposed opposite the source on the second surface of the gantry to detect the radiation transmitted through the subject.

The gantry may have a polygonal or circular column shape with both ends open.

At least parts under the first surface of the gantry may be connected to rails provided parallel to a transport direction of the subject.

Rail connectors may be provided at lower parts of the first surface of the gantry to move the gantry on rails in a direction parallel to a transport direction of the subject.

The modules may further include a second gantry disposed adjacent to the gantry to provide an internal space through which the subject is transported, and having a first surface corresponding to an outer circumferential surface and a second surface corresponding to an inner circumferential surface, a second source disposed on the second surface of the second gantry to generate and emit radiation toward the subject, and a second detector disposed opposite the second source on the second surface of the second gantry to detect the radiation transmitted through the subject.

The source of the gantry and the second source of the second gantry may be offset by an angle greater than at least 20° around a central axis of the internal space on a plane perpendicular to a transport direction of the subject.

At least one step portion may be formed at a side of the gantry.

At least one step portion may be formed at a side of the gantry, at least one second step portion may be formed at a side of the second gantry, and the step portion and the second step portion may have complementary shapes so as to fit together.

A shielding member for shielding radiation leakage from the internal space may be disposed on at least a partial surface of the step portion.

Sides of the gantry and the second gantry may be disposed adjacent to each other, and shielding members for shielding radiation leakage from the internal space may be disposed on at least partial surfaces of the step portion and the second step portion to seal a gap between the step portion and the second step portion.

Coupling members may be assembled on the first surfaces of the gantry and the second gantry to connect the internal space of the gantry to the internal space of the second gantry.

A pin may be formed on at least a part of a side of the gantry in a direction parallel to a transport direction of the subject.

A pin may be formed on at least a part of a side of the gantry in a direction parallel to a transport direction of the subject, and a hole may be formed in at least a part of a side of the second gantry, corresponding to the pin, to accommodate the pin.

The detector may have an arc shape to maintain an equal distance from the source to every part of the detector.

The detector may include a collimator including a shieling part for shielding the radiation, and a slit for transmitting the radiation, and cells for detecting the transmitted radiation.

When a width of the gantry in a transport direction of the subject is denoted by w, a distance between the source and the cells is denoted by L, a distance between the source and the collimator is denoted by I, a distance between the collimator and the cells is denoted by d, and a width of the cells is denoted by s, the collimator of the detector may be configured to satisfy (Inequality) w/L≤(w−s/2)/l [where L≥I+d].

According to an aspect of the present invention, there is provided a computed tomography (CT) device for capturing a tomographic image along a transport direction of a subject, the CT device including a front portion provided to allow entrance of the subject and to shield radiation, a scanner for providing an internal space to allow transportation of the subject from the front portion and to scan the subject, a rear portion provided to allow exit of the subject after passing through the scanner and to shield radiation, wherein the scanner is configured by connecting a plurality of modules to each other in a direction parallel to the transport direction of the subject, wherein each module includes a gantry for providing an internal space through which the subject is transported, and having a first surface corresponding to an outer circumferential surface and a second surface corresponding to an inner circumferential surface, a source disposed on the second surface of the gantry to generate and emit radiation toward the subject, and a detector disposed opposite the source on the second surface of the gantry to detect the radiation transmitted through the subject.

At least parts under the first surface of each module may be connected to rails provided parallel to the transport direction of the subject.

A space created between a specific module and other modules by moving the specific module along an extension direction of the rails is provided as a maintenance space.

The specific module may be removed from the rails by disassembling rail connectors provided between the specific module and the rails.

The sources of a pair of adjacent modules may be offset by an angle greater than at least 20° on a plane perpendicular to the transport direction of the subject.

Step portions may be formed at sides of the pair of adjacent modules in complementary shapes so as to fit together.

Shielding members for shielding radiation leakage from the internal space may be disposed on at least partial surfaces of the step portions to seal a gap between the step portions.

A pin and hole may be formed on sides of the pair of adjacent modules, and the pin of one module may be inserted into the hole of the other module to prevent disconnection between the gantries of the pair of modules.

The detector may include a collimator including a shieling part for shielding the radiation, and a slit for transmitting the radiation, and cells for detecting the transmitted radiation and, when a distance between the cells of Nth and (N−1)th modules among the plurality of modules is denoted by w, a distance between the source and the cells of the Nth module is denoted by L, a distance between the source and the collimator of the Nth module is denoted by I, a distance between the collimator and the cells of the Nth module is denoted by d, and a width of the cells of the Nth module is denoted by s, the collimator of the detector of the Nth module may be configured to satisfy (Inequality) w/L≤(w−s/2)/l [where L≥I+d].

### Advantageous Effects

According to the present invention configured as described above, a computed tomography (CT) device capable of freely assembling a plurality of modules to minimize the effect of scatter noise between fan beams, and modules for the CT device, may be provided.

According to the present invention, the modules may be suitably combined in consideration of factors such as the purpose of use of the device, scanning speed, image resolution, and subject size.

According to the present invention, the entire device may be conveniently maintained by replacing or repairing only a specific module.

According to the present invention, a source and detector may be disposed in a gantry to minimize external radiation leakage, and shielding efficiency between modules may be improved.

However, the scope of the present invention is not limited to the above effects.

### DESCRIPTION OF THE DRAWINGS

FIG. 1 is a schematic view of a computed tomography (CT) device according to an embodiment of the present invention.

FIG. 2 is a schematic view of a module for a CT device, according to an embodiment of the present invention.

FIG. 3 is a schematic view of a scanner of a CT device, according to an embodiment of the present invention.

FIG. 4 is a plan view showing the arrangement of modules for a CT device, according to an embodiment of the present invention.

FIG. 5 is a schematic view showing a switching operation sequence of modules for a CT device, according to an embodiment of the present invention.

FIG. 6 is a perspective view showing the arrangement of modules for a CT device, according to another embodiment of the present invention.

FIG. 7A is a perspective view, FIG. 7B is an A-A′ cross-sectional view, and FIG. 7C is a B-B′ cross-sectional view of some modules of a CT device, according to an embodiment of the present invention.

FIG. 8 is an enlarged view of portion C of FIGS. 7A, 7B, 7C.

FIG. 9 is perspective view showing a pin structure of modules for a CT device, according to an embodiment of the present invention.

FIG. 10 is a schematic view of radiation collimators of a source and detector, according to an embodiment of the present invention.

FIG. 11 is a schematic view showing a structure for preventing radiation scatter from an adjacent module, according to an embodiment of the present invention.

FIG. 12 is a schematic view showing a maintenance process of a specific module, according to an embodiment of the present invention.

FIG. 13 is a schematic view showing a removal/replacement process of a specific module, according to an embodiment of the present invention.

FIG. 14 includes images acquired according to a comparative example and a test example of the present invention.

### Explanation of Reference Numerals

10: Module
11: Gantry
11-1a, 11-2a: Step portions
12: Source
15: Detector
17: Shielding members
70: Rails
72: Rail connectors
100: Scanner
200: Front portion
300: Rear portion
400: Subject
500: Transport unit
CM1, CM2: Coupling members
G: Gap
IT: Internal space, Tunnel space
OT: External space
P1: Pins
P2: Holes

### MODE OF THE INVENTION

The following detailed description of the invention will be made with reference to the accompanying drawings illustrating specific embodiments of the invention by way of example. These embodiments will be described in sufficient detail such that the invention may be carried out by one of ordinary skill in the art. It should be understood that various embodiments of the invention are different but do not need to be mutually exclusive. For example, a specific shape, structure, or characteristic described herein in relation to an embodiment may be implemented as another embodiment without departing from the scope of the invention. In addition, it should be understood that positions or arrangements of individual elements in each disclosed embodiment may be changed without departing from the scope of the invention. Therefore, the following detailed description should not be construed as being restrictive and, if appropriately described, the scope of the invention is defined only by the appended claims and equivalents thereof. In the drawings, like reference numerals denote like functions, and lengths, areas, thicknesses, and shapes may be exaggerated for convenience's sake.

Hereinafter, the present invention will be described in detail by explaining embodiments of the invention with reference to the attached drawings, such that one of ordinary skill in the art may easily carry out the invention.

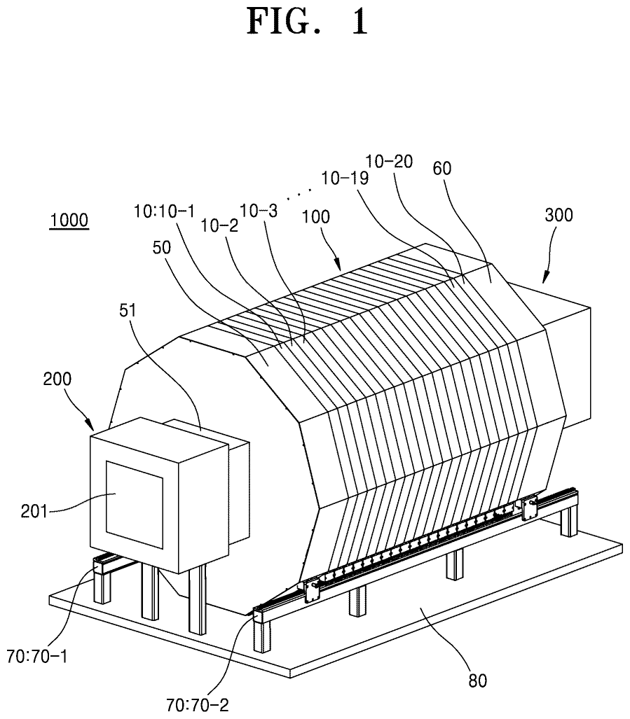

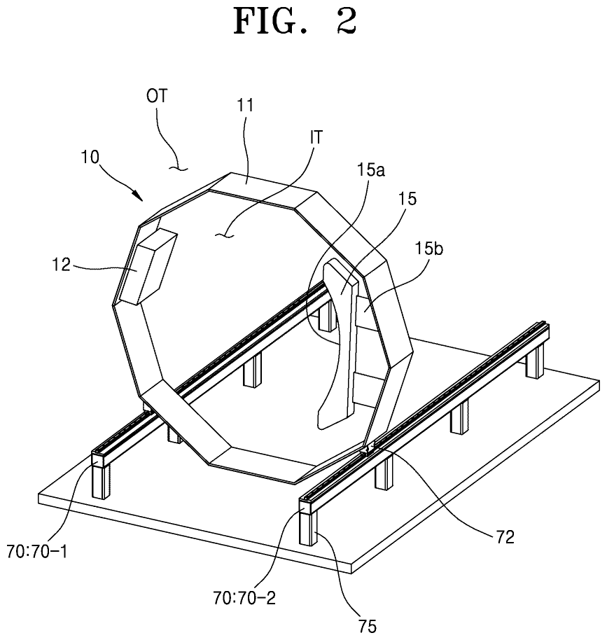

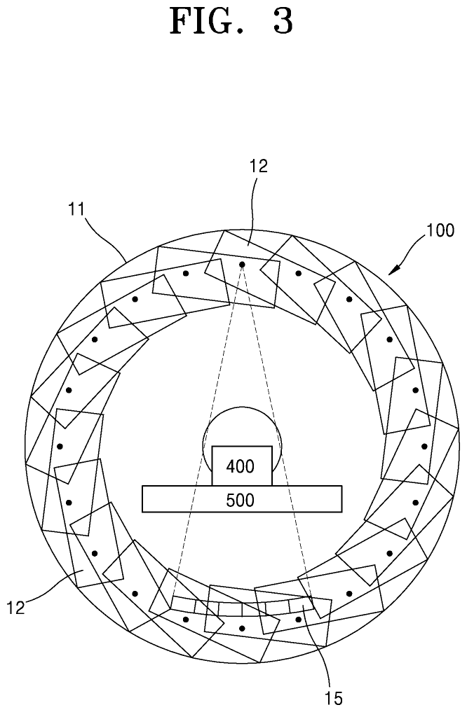

FIG. 1 is a schematic view of a computed tomography (CT) device 1000 according to an embodiment of the present invention. FIG. 2 is a schematic view of a module for a CT device, according to an embodiment of the present invention. FIG. 3 is a schematic view of a scanner of a CT device, according to an embodiment of the present invention.

Referring to FIG. 1, the CT device 1000 according to an embodiment of the present invention may include a scanner 100, a front portion 200, and a rear portion 300. The CT device 1000 may further include rails 70 and a base 80.

The scanner 100 may scan a subject 400. The scanner 100 may provide an internal space IT [or tunnel space IT] along a transport direction of the subject 400 (or the Z-axis direction). The subject 400 may be scanned both internally and externally while moving along the internal space IT.

The front portion 200 may provide an inlet 201 through which the subject 400 enters the CT device 1000. The inlet may be connected to the internal space IT of the scanner 100. The front portion 200 may include a certain radiation shielding means to prevent the radiation emitted from the scanner 100 toward the subject 400 from leaking outside.

The rear portion 300 may provide an outlet (not shown) through which the subject 400 having passed through the scanner 100 exits. The outlet may be connected to the internal space IT of the scanner 100. The rear portion 300 may include a certain radiation shielding means to prevent the radiation emitted from the scanner 100 toward the subject 400 from leaking outside.

Referring to FIGS. 1 to 3, the scanner 100 [or a gantry 11 of a module 10] according to the present invention is characterized by being stationary rather than rotary. A rotary gantry requires separate means for rotation, which results in a complex structure and increased manufacturing costs. Additionally, when continuous scanning is required, e.g., at airport security checkpoints, constantly rotating the gantry is inconvenient, and frequent failures occur due to the weight of the gantry during rotation. On the other hand, by adopting the stationary scanner 100 [or gantry 11], the present invention may provide the CT device 1000 capable of reducing manufacturing costs and improving durability.

The subject 400 is placed on a transport unit 500, and the transport unit 500 transports the subject 400 in the Z-axis direction under the control of a controller. The following description assumes that a plane perpendicular to the transport direction of the subject 400, or a plane occupied by the gantry 11 of the module 10, is referred to as the XY-plane, and that a horizontal direction is referred to as the X-axis direction whereas a vertical direction is referred to as the Y-axis direction.

The rails 70 may be provided to extend in a direction parallel to the transport direction of the subject 400 (or the Z-axis direction). The rails 70 may include one or more rails 70-1 and 70-2. The rails 70 may be provided to support the scanner 100 [or a plurality of modules 10] and, at the same time, to allow the modules 10 of the scanner 100 to move in the Z-axis direction.

Rail connectors 72 may be interposed between the scanner 100 [or the plurality of modules 10] and the rails 70. The rail connectors 72 may be connected to lower parts of the scanner 100 [or the plurality of modules 10/gantries 11], to support the scanner 100 [or the plurality of modules 10] on the rails 70 and, at the same time, to move in the Z-axis direction on the rails 70.

The base 80 may be provided in a plate shape supporting the CT device 1000. Rail support frames 75 may be provided on the base 80 in a vertical direction, and the rails 70 may be disposed on the rail support frames 75. Support frames (not shown) supporting the front portion 200/rear portion 300 may be additionally disposed on the base 80.

The CT device 1000 of the present invention is characterized by configuring the scanner 100 by connecting a plurality of modules 10: 10-1, 10-2, 10-3, . . . 10-19, and 10-20. Each of the modules 10: 10-1 to 10-20 may include a gantry 11 with a certain width (Z-axis direction width), a source 12, and a detector 15. In particular, the gantry 11 has a width of several to tens of centimeters (cm), and thus may be easily manufactured. Conventional CT devices include a gantry with a solid plate structure and a length of several meters (m), and the large size thereof may cause difficulties in mold fabrication. Additionally, when an error occurs inside, disassembling and replacing the entire gantry is very cumbersome, and maintenance costs increase. However, according to the CT device 1000 of the present invention, even when an error occurs in a specific gantry 11, source 12, or detector 15, only the module 10 including the faulty component may be removed and replaced, thereby enabling very convenient maintenance.

The gantry 11 may have a column shape with both ends open to provide the internal space IT. Although FIG. 2 shows the gantry 11 with a decagonal column shape, the gantry 11 may also have a polygonal column shape such as an octagonal or decagonal column, or a circular column shape. In the following description, the outer circumferential surface of the gantry 11 may also be referred to as a first surface, and the inner circumferential surface thereof may also be referred to as a second surface.

The Z-axis direction width of the gantry 11 is several to tens of cm, and the plurality of modules 10: 10-1 to 10-20 may be connected along the Z-axis direction to configure the scanner 100 with a length of several m. For example, twenty modules 10: 10-1 to 10-20 with a width of approximately 90 mm may be connected to configure the scanner 100 with a length of approximately 2 m. The gantry 11 may be made of a metal material to maintain rigidity.

Meanwhile, housings 50 and 60 may be further connected to both ends of the modules 10 configuring the scanner 100. Each housing 50 or 60 may be configured in the form of the module 10 with one side of the gantry 11 covered. Certain connectors 51 may be provided on the covered sides of the housings 50 and 60 and connected to the inlet of the front portion 200/the outlet of the rear portion 300.

The source 12 may be disposed on the inner circumferential surface (second surface) of the gantry 11. The source 12 may generate and emit radiation toward the subject 400. For example, the radiation may be X-rays in the form of a fan beam. A connecting member (not shown) may be interposed between the inner circumferential surface of the gantry 11 and the source 12 to adjust the angle of the radiation emitted from the source 12 into the internal space IT.

In general, a source is disposed outside a gantry, and certain through-holes are formed in the gantry to allow X-rays to be emitted from the source. Disposing the source outside the gantry enables better utilization of the internal space of the gantry and increases the distance from the source to a detector, thereby improving image resolution. However, because some of the X-rays are scattered/reflected rather than passing through the through-holes, radiation may leak outside the gantry.

Therefore, to solve the above problem, the present invention may minimize the radiation leakage to an external space OT by disposing the source 12 inside the gantry 11. The airtightness of the internal space IT of the gantry 11 from the external space OT may be further improved using step portions 11-1a and 11-2a of the gantry 11, shielding members 17, and pins P1/holes P2, which will be described below.

The detector 15 may be disposed opposite the source 12 on the inner circumferential surface (second surface) of the gantry 11. The detector 15 may detect the radiation transmitted through the subject 400. For example, the detector 15 may be configured as a photodetector, but is not limited thereto as long as a means capable of detecting radiation is used.

Meanwhile, conventionally, a device in which an array of X-ray tubes serving as multiple sources are provided in a curved shape and multiple detectors are provided in a linear shape has been disclosed. This device has a simple structure that sequentially emits X-rays from the multiple sources, but suffers from low scanning speed and image resolution. Another conventional device including detectors in an ‘L’ or ‘U’ shape to improve spatial efficiency has also been proposed. However, image distortion occurs because the distance from the source to the detector varies. Although the distortion is corrected using software, noise resulting from the correction still remains.

Therefore, according to an embodiment of the present invention, the detector 15 is characterized by an arc shape. In other words, a side surface 15a of the detector 15 facing the source 12 may have an arc or curved shape. In particular, all parts of the side surface 15a may be spaced equally from the source 12 to form an arc shape. The side surface 15a of the detector 15 may have an arc shape that maintains an equal distance from the radiation-emitting part of the source 12. Because radiation may be detected at the side surface 15a of the detector 15 within the same distance, image distortion does not occur.

Meanwhile, the detector 15 with the arc-shaped side surface 15a may not be easily disposed directly on the inner circumferential surface of the gantry 11. That is, because the arc-shaped side surface 15a of the detector 15 and the inner circumferential surface of the gantry 11 may not have the same curvature, auxiliary supports 15b may be installed on the inner circumferential surface of the gantry 11, and the detector 15 may be connected to the auxiliary supports 15b to allow the arc-shaped side surface 15a to face the source 12.

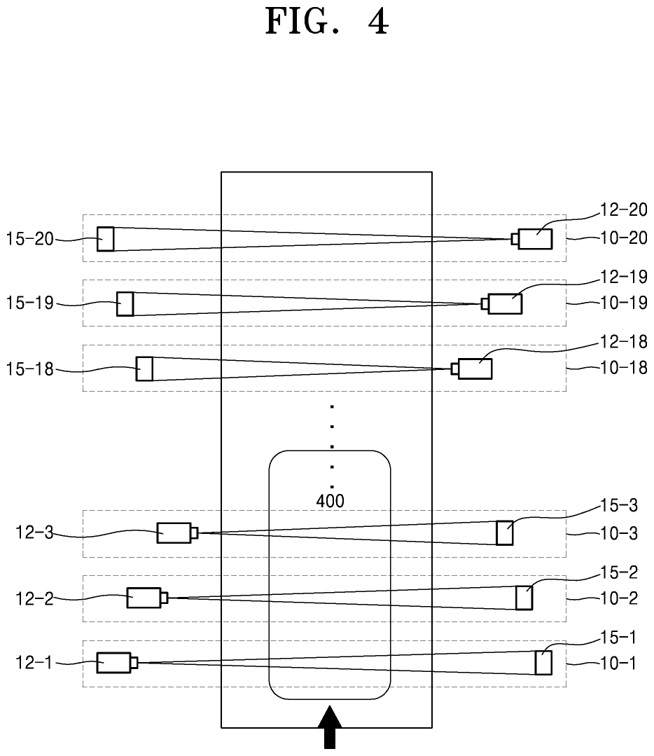

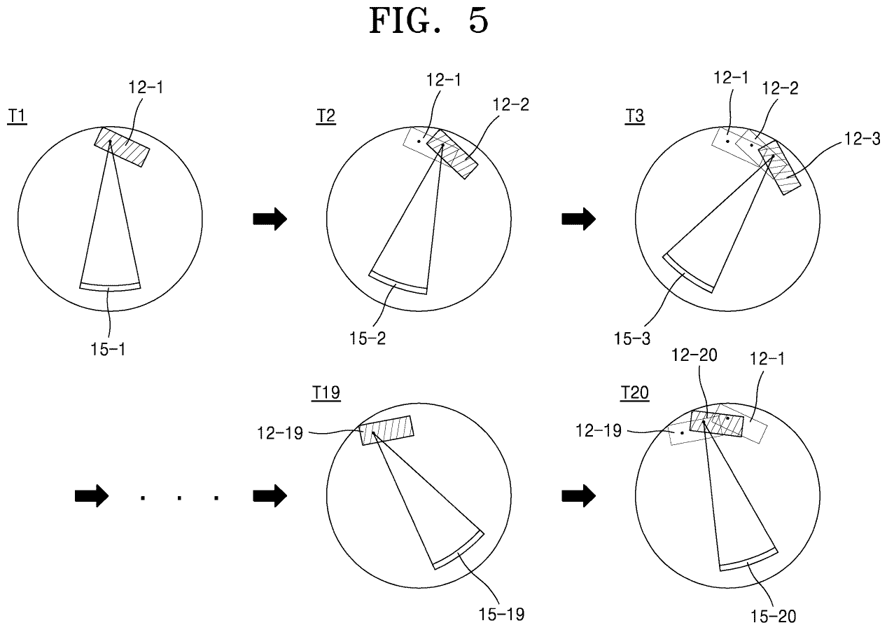

FIG. 4 is a plan view showing the arrangement of modules 10 for a CT device, according to an embodiment of the present invention. FIG. 5 is a schematic view showing a switching operation sequence of the modules 10 for a CT device, according to an embodiment of the present invention.

Referring to FIG. 4, each of the modules 10: 10-1 to 10-20 may acquire a tomographic image. As an embodiment, twenty modules 10: 10-1 to 10-20 are shown. The positions of the sources 12: 12-1 to 12-20 and detectors 15: 15-1 to 15-20 in the modules 10: 10-1 to 10-20 may be different. XY-plane tomographic images may be acquired while the subject 400 placed on the transport unit 500 is being transported in the Z-axis direction under motor control.

Meanwhile, FIG. 3 shows twenty sources 12: 12-1 to 12-20 overlapped on the XY plane. The sources 12: 12-1 to 12-20 may be disposed on the inner circumferential surface of the gantries 11: 11-1 to 11-20 at 18° intervals from each other. For example, the source 12-1 of the first gantry 11-1 may be disposed at a position of 0°, the source 12-2 of the second gantry 11-2 may be disposed at 18°, the source 12-3 of the third gantry 11-3 may be disposed at 36°, . . . , and the source 12-20 of the twentieth gantry 11-20 may be disposed at 342°. The detectors 15: 15-1 to 15-20 may be disposed opposite the sources 12: 12-1 to 12-20, respectively.

FIG. 5 is a schematic view showing a switching operation sequence of the modules 10 for a CT device, according to an embodiment of the present invention.

Referring to FIG. 5, the sources 12: 12-1 to 12-20 may sequentially emit radiation in periods T1 to T20, and the detectors 15: 15-1 to 15-20 may detect the radiation transmitted through the subject 400, thereby acquiring tomographic images. For example, fan-beam X-rays emitted from the source 12-1 of the first module 10-1 may be transmitted through the subject 400 and detected by the detector 15-1 in period T1, fan-beam X-rays emitted from the source 12-2 of the second module 10-2 may be transmitted through the subject 400 and detected by the detector 15-2 in period T2, and fan-beam X-rays emitted from the source 12-3 of the third module 10-3 may be transmitted through the subject 400 and detected by the detector 15-3 in period T3. The detection process of the detectors 15 continues sequentially until period T20.

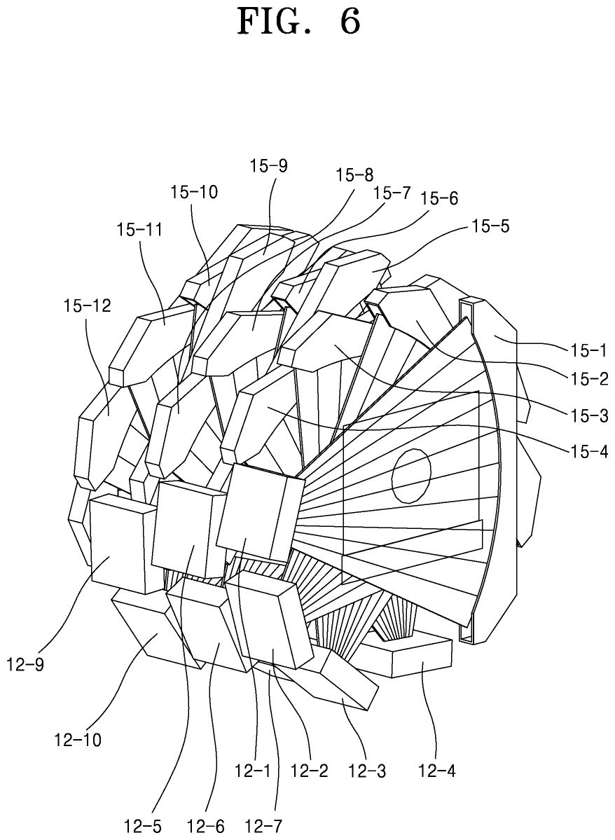

FIG. 6 is a perspective view showing the arrangement of modules for a CT device, according to another embodiment of the present invention.

Meanwhile, the sources 12/detectors 15 of adjacent modules 10 may not necessarily be offset by the same angle of 18° as illustrated in FIGS. 3 to 5. When adjacent modules 10 emit radiation toward the subject 400 in an almost parallel manner, noise may occur in the detectors 15 of the adjacent modules 10 due to the radiation reflected or scattered from the subject 400.

Therefore, the sources 12/detectors 15 of adjacent modules 10 may be offset by 20° or more. Referring to FIG. 6, when the source 12-1 of the first module 10-1 is initially disposed at 0°, the source 12-2 of the second module 10-2 may be disposed at 30°, the source 12-3 of the third module 10-3 may be disposed at 60°, and the source 12-4 of the fourth module 10-4 may be disposed at 90°. Then, the source 12-5 of the fifth module 10-5 may be disposed at 10°, the source 12-6 of the sixth module 10-6 may be disposed at 40°, the source 12-7 of the seventh module 10-7 may be disposed at 70°, and the source 12-8 of the eighth module 10-8 may be disposed at 100°, so as to be shifted by 10° from the sources of the first to fourth modules 10-1 to 10-4. In the same manner, the source 12-9 of the ninth module 10-9 may be disposed at 20°, the source 12-10 of the tenth module 10-10 may be disposed at 50°, the source 12-11 of the eleventh module 10-11 may be disposed at 80°, and the source 12-12 of the twelfth module 10-12 may be disposed at 110°, so as to be shifted by 10° from the sources of the fifth to eighth modules 10-5 to 10-8. This arrangement may be modified to optimize the scanning of the subject 400 placed directly on the transport unit 500.

Offsetting the sources 12/detectors 15 of adjacent modules 10 by 20° or more may include offsets of 20° or more from opposite sides, i.e., at angles ranging from 20° to 160° and from 200° to 340°.

As another example, the sources 12: 12-1 to 12-20 are not necessarily driven sequentially from period T1 to period T20 in FIG. 5 and may be driven in a different order to minimize scatter noise.

According to the present invention, the number of modules 10 may be freely changed depending on the purpose of CT scanning, a subject to be scanned, and a desired image resolution. In addition, the positions of the sources 12/detectors 15 of the modules 10 may be individually changed. Therefore, the CT device 1000 may be very easily adapted for various purposes.

Meanwhile, although each module 10 includes one source 12 and one detector 15 in the above description, each module 10 may also include multiple pairs of sources 12 and detectors 15.

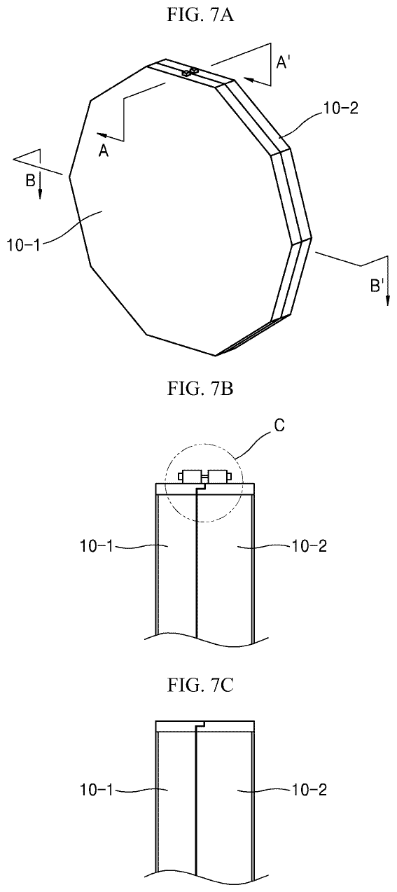

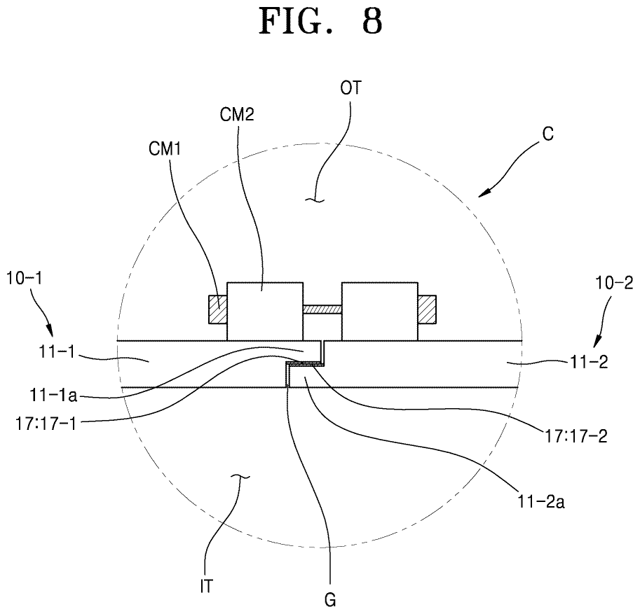

FIG. 7A is a perspective view, FIG. 7B is an A-A′ cross-sectional view, and FIG. 7C a B-B′ cross-sectional view of some modules 10-1 and 10-2 of the CT device 1000, according to an embodiment of the present invention. FIG. 8 is an enlarged view of portion C of FIGS. 7A, 7B, and 7C.

Because the scanner 100 of the present invention is not a single integrated cylindrical structure but is composed of a plurality of modules 10, a gap G may occur between the modules 10. Radiation may leak through this gap G. Therefore, the assembly tolerance of the gantries 11 of the modules 10 needs to be minimized.

Referring to FIGS. 7B and 7C, and FIG. 8, the modules 10-1 and 10-2 may be connected along the Z-axis direction by bringing their facing side surfaces into contact. The gantries 11-1 and 11-2 of the adjacent modules 10-1 and 10-2 may be coupled to each other by assembling coupling members CM1 and CM2 on the outer circumferential surfaces (first surfaces) of the gantries 11-1 and 11-2. The coupling members CM1 and CM2 may use any known coupling means such as bolts, nuts, rivets, or screws without limitation. When the gantries 11-1 and 11-2 are coupled to each other, the internal space IT of each gantry 11-1 or 11-2 may be connected to the other and gradually expand in the Z-axis direction.

Meanwhile, when facing side surfaces of the gantries 11-1 and 11-2 are vertical, there is a high possibility of radiation leakage through the linearly formed gap G even after coupling with the coupling members CM1 and CM2. Therefore, according to the present invention, step portions 11-1a and 11-2a may be formed on the side surfaces of the gantries 11-1 and 11-2. The step portions 11-1a and 11-2a may have a single-step or multi-step structure. The first step portion 11-1a of the first gantry 11-1 and the second step portion 11-2a of the second gantry 11-2 may have complementary shapes so as to fit together. Alternatively, the step portions 11-1a and 11-2a may have interlocking concave-convex shapes. By providing the step portions 11-1a and 11-2a on the side surfaces of the gantries 11-1 and 11-2, the gap G may be formed in a winding rather than linear manner. That is, the gap G may extend along a longer path than a vertical straight line. In addition, because the gap G includes a horizontal part which is not vertically straight, radiation leakage may be effectively blocked.

Furthermore, because the side surfaces of the gantries 11-1 and 11-2 are joined together by the step portions 11-1a and 11-2a, assembly tolerance may be minimized, and a firmer connection may be achieved compared to using only the coupling members CM1 and CM2. In particular, when the gantries 11-1 and 11-2 have a polygonal column shape rather than a circular column shape, the angular corners of the polygon may help ensure precise alignment and more robust coupling between the gantries 11-1 and 11-2.

Referring back to FIG. 8, shielding members 17: 17-1 and 17-2 may be further provided on at least partial surfaces of the step portions 11-1a and 11-2a. For example, the shielding members 17 may be made of a lead (Pb) sheet or a known shielding material. The shielding members 17 may be disposed on the step portions 11-1a and 11-2a to provide a tighter seal and prevent the formation of the gap G. By disposing the shielding members 17 on horizontal parts of the gap G, shielding efficiency may be improved compared to disposing them on vertical parts of the gap G. As described above, according to the present invention, radiation may be double-shielded by the gantries 11 through the step portions 11-1a and 11-2a and the shielding members 17.

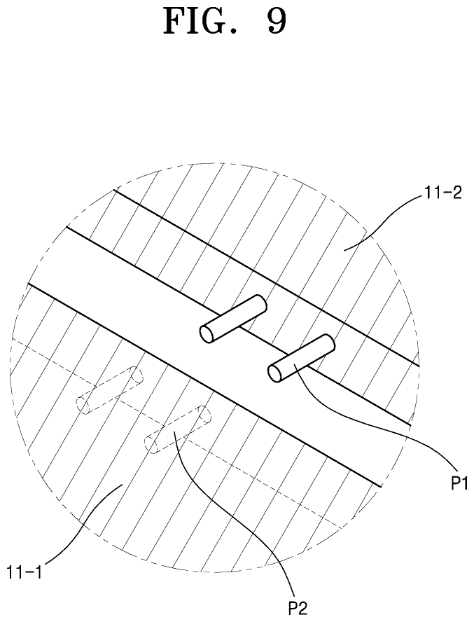

FIG. 9 is a perspective view showing a pin structure of the modules 10 for a CT device, according to an embodiment of the present invention. FIG. 9 is an enlarged perspective view of portion C in FIG. 7B. For convenience of explanation, FIG. 9 omits the step portions 11-1a and 11-2a and shows only the side surfaces of the gantries 11-1 and 11-2.

The gantries 11 of the modules 10 may undergo a certain degree of warping or deformation due to its own weight. Such deformation may increase the gap G and raise the risk of radiation leakage. Therefore, pins P1 may be formed at a side of the gantry 11-2 in the Z-axis direction. In addition, holes P2 into which the pins P1 are inserted may be formed at a side of the adjacent gantry 11-1. Although FIG. 9 shows that the holes P2 are formed in the first gantry 11-1 and the pins P1 are formed on the second gantry 11-2, the pins P1 and holes P2 may be simultaneously formed at corresponding positions of the first and second gantries 11-1 and 11-2.

Because the pins P1 are inserted into the holes P2 to couple the gantries 11-1 and 11-2 to each other, the gantries 11-1 and 11-2 may be more securely coupled and fixed without misalignment, and the size of the gap G and assembly tolerance may be minimized to further prevent external radiation leakage.

Meanwhile, the pins P1 and holes P2 may be formed on a plurality of parts of the side surface of each gantry 11. For example, ten sets of pins P1 and holes P2 may be formed at the vertices of a decagonal-column-shaped gantry 11. As another example, a plurality of pins P1 and holes P2 may be formed on the side surface of a circular-column-shaped gantry 11 at certain intervals. In particular, because the circular-column-shaped gantry 11 may be misaligned along the circumferential direction even when the step portions 11-1a and 11-2a are formed, the pins P1/holes P2 may more firmly fix the position of the gantry 11.

Radiation doses were measured according to a comparative example and a test example of the present invention. In a CT device according to the comparative example, step portions and shielding members are not provided and facing side surfaces of gantries are vertical. In the CT device 1000 according to the test example of the present invention, the step portions 11-1a and 11-2a are provided and the shielding members 17:17-1 and 17-2 made of a lead sheet are provided on the horizontal parts of the gap G between the step portions 11-1a and 11-2a.

The radiation doses were measured at five positions on the CT devices according to the comparative example and the test example of the present invention: the front portion 200, the rear portion 300, the front of the scanner 100, the rear of the scanner 100, and the top of the scanner 100. In the comparative example, the measured radiation doses at the five positions were approximately 5 to 9 times higher than the natural background radiation dose. On the other hand, in the test example of the present invention, the measured radiation doses at the five positions were approximately 1 to 2 times higher than the natural background radiation dose. It is shown that the radiation leakage in the test example of the present invention is reduced to 20% or less of that in the comparative example.

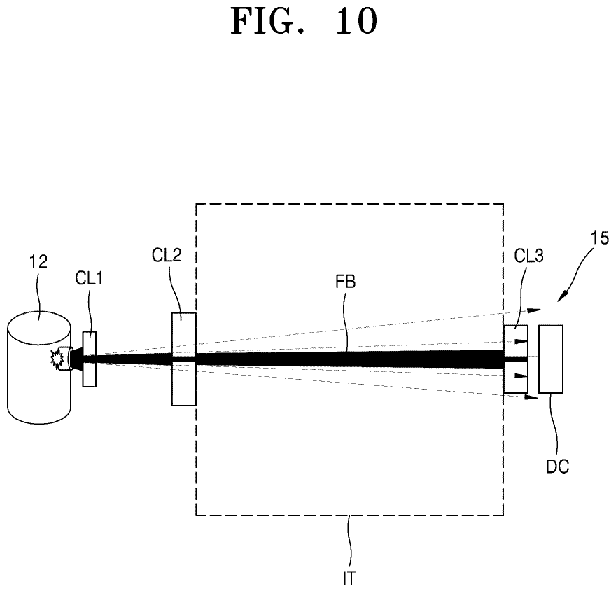

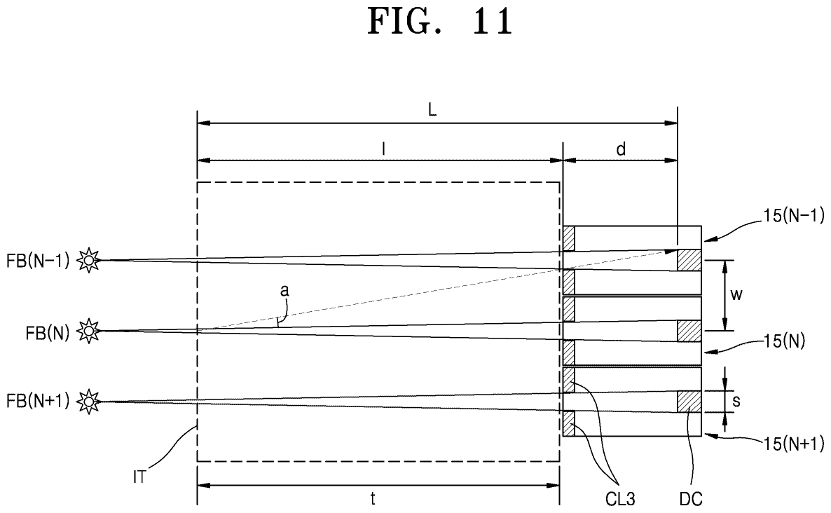

FIG. 10 is a schematic view of radiation collimators CL1 to CL3 of the source 12 and detector 15, according to an embodiment of the present invention. FIG. 11 is a schematic view showing a structure for preventing radiation scatter from an adjacent module 10, according to an embodiment of the present invention.

Radiation emitted from the focal spot of an anode target in an X-ray tube refers to a form of light that spreads through space in a radial direction. In order to shape X-rays exiting through a window into a desired form of beam at a target point, a collimator for forming the X-rays into a fan beam is required. To minimize scattered radiation in security screening equipment, it is most effective to provide a collimator at each stage where the fan beam is projected. However, the system needs to be designed in consideration of factors such as manufacturing cost, assembly complexity, and the ease of collimator alignment. When scattered radiation occurs, the quality of the X-ray beam may be degraded, and image quality may be negatively affected.

Initially, referring to FIG. 10, three main parts may be provided with the collimators CL1 to CL3 to reduce scattered radiation. Because a radiation beam FB is emitted from the source 12 at a wide angle, the first collimator (fan collimator) CL1 may primarily reduce the angle. Then, immediately before the radiation beam FB enters the internal space IT [or tunnel space IT], the second collimator (pre-collimator) CL2 may secondarily reduce the angle. Subsequently, for the radiation beam FB transmitted through the subject 400, the third collimator (detector collimator) CL3 may block the radiation beam FB incident on the side walls and transmit the radiation beam FB only through a slit formed in the middle. As such, only the radiation beam FB that directly enters the front of cells DC of the detector 15 may be detected.

Meanwhile, according to the present invention, because a plurality of modules 10 are provided, scatter of radiation beams may have an effect between adjacent modules 10. Therefore, a design capable of reducing the noise of the radiation beams FB and optimizing the detection at the cells DC will now be described with reference to FIG. 11.

Referring to FIG. 11, because the detector 15 includes an arc-shaped side surface 15a as described above, it may be assumed that the distances from the source 12 to all the cells DC of the detector 15 are equal. The following description is provided for (N−1)th, Nth, and (N+1)th gantries 11.

The (N−1)th, Nth, and (N+1)th gantries 11 may be spaced apart by the same distance w. The distance w may correspond to the Z-axis direction width w of each gantry 11. The distance between the source 12 and the cells DC is denoted by L, the distance between the source 12 and the collimator CL3 of the detector 15 is denoted by I, the distance between the collimator CL3 and the cells DC is denoted by d, and the width of the cells DC is denoted by s.

In this case, radiation scatter noise from an adjacent module 10 has the smallest incident angle at the farthest distance I+d in the tunnel space IT. When the distance decreases, the incident angle may increase. Therefore, to minimize incident noise, the system needs to be designed to block noise incident from the farthest distance I+d.

Radiation directed to the detector 15 through the slit formed in the collimator CL3 needs to be incident in a manner that matches or covers the width s of the cells DC. Radiation directed to other areas needs to be shielded and blocked by a shieling part of the collimator CL3.

The amount by which the radiation noise diagonally incident from the adjacent module 10 is blocked may vary depending on factors such as the shieling part thickness and slit width of the collimator CL3. When the collimator CL3 moves closer to the source 12, an incident angle a of the radiation diagonally incident from the adjacent module 10 may increase, thereby improving the shielding effect. However, because the slit width also needs to be reduced, manufacturing costs may rise. On the other hand, when the collimator CL3 moves closer to the cells DC, the slit may be widened to approach the width s of the cells DC, reducing manufacturing costs. However, to block the scattered radiation incident in a diagonal direction, a shieling part thickness d of the collimator CL3 needs to be increased, which ultimately raises manufacturing costs.

Therefore, to block even a radiation beam with the smallest noise angle a, the relationship between the position and thickness of the collimator CL3 considering I and d may be expressed as the following inequality.

$$( Inequality ) w / L \leq ( w - s / 2 ) / I ( where L \geq I + d )$$

Therefore, the collimator CL of the detector 15 in the Nth module may be designed to satisfy the Inequality. The greater the distance between the collimator CL3 and the cells DC, the more effectively the radiation beam may be blocked. The inequality may be understood as defining the minimum distance.

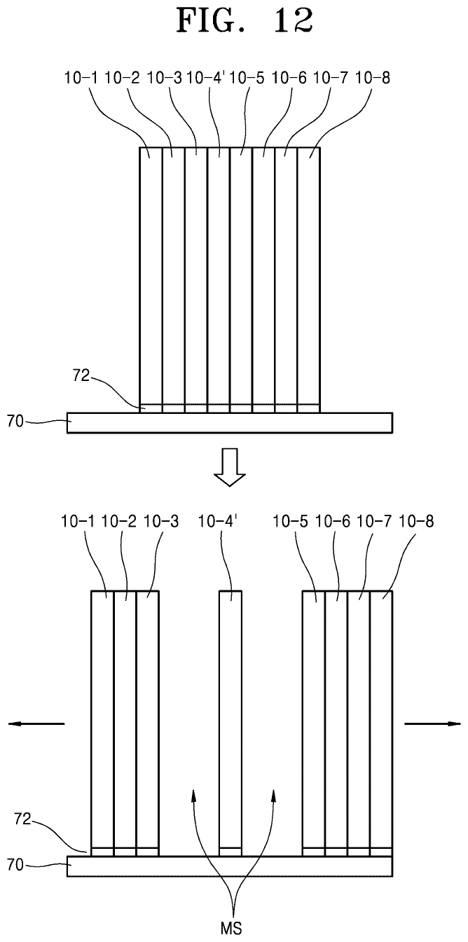

FIG. 12 is a schematic view showing a maintenance process of a specific module, according to an embodiment of the present invention.

As described above, a plurality of modules 10 may be provided with the rail connectors 72 at lower parts of the outer circumferential surface (first surface) to move along the rails 70 in the Z-axis direction via the rail connectors 72.

Initially, as shown in the upper part of FIG. 12, a faulty module 10-4′ may be identified. Then, the modules 10-1 to 10-3, and 10-5 to 10-8 adjacent to both sides of the specific module 10-4′ may be moved along the extension direction of the rails 70 (or the Z-axis direction). The modules 10-1 to 10-3 coupled in front of the module 10-4′ may be moved forward, while the modules 10-5 to 10-8 coupled behind the module 10-4′ may be moved rearward. Before moving the modules 10-1 to 10-3, and 10-5 to 10-8, the coupling members CM1 and CM2 [see FIG. 8] of the modules 10-3 and 10-5 adjacent to the front/rear of the module 10-4′ may be disassembled.

Subsequently, as shown in the lower part of FIG. 12, when the modules 10-1 to 10-3 are moved forward along the rails 70, a maintenance space MS may be created between the modules 10-3 and 10-4′. Likewise, when the modules 10-5 to 10-8 are moved rearward along the rails 70, the maintenance space MS may also be created between the modules 10-5 and 10-4′. Through this maintenance space MS, a user may access the interior of the module 10-4′ to perform maintenance work.

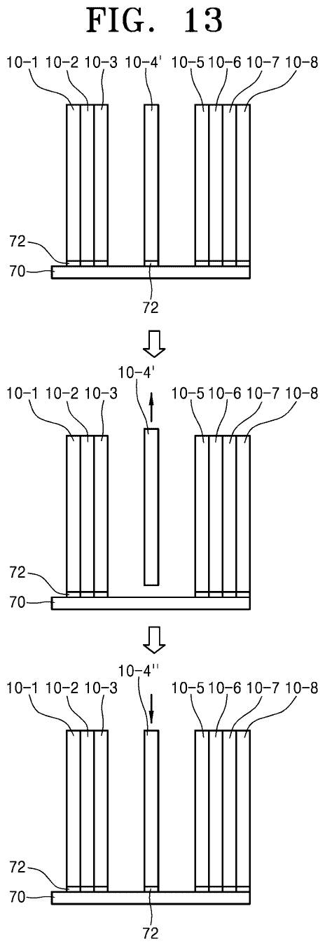

FIG. 13 is a schematic view showing a removal/replacement process of a specific module, according to an embodiment of the present invention.

Initially, as shown in the upper part of FIG. 13, a faulty module 10-4′ may be identified. Then, the modules 10-1 to 10-3, and 10-5 to 10-8 may be moved forward/rearward as in FIG. 12

Subsequently, as shown in the middle part of FIG. 13, the specific module 10-4′ may be removed from the rails 70. The rail connectors 72 for connecting the specific module 10-4′ to the rails 70 may be disassembled, and then the specific module 10-4′ may be lifted and removed.

Then, as shown in the lower part of FIG. 13, a new module 10-4″ may be positioned on the rails 70 and then the rail connectors 72 may be assembled to connect the module 10-4″ onto the rails 70. Thereafter, the modules 10-1 to 10-3, and 10-5 to 10-8 may be brought back into close contact with the module 10-4″ and then the coupling members CM1 and CM2 may be reassembled.

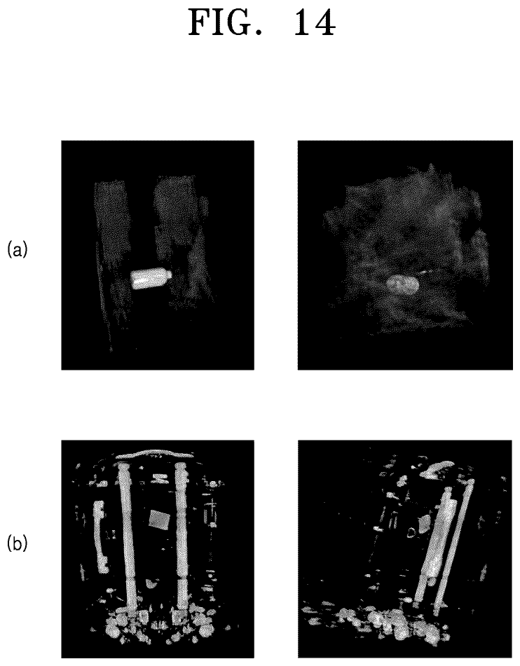

FIG. 14 includes images acquired according to a comparative example and a test example of the present invention.

(a) of FIG. 14 includes front and side images of a bag, which are captured by a CT device equipped with nine sources/detectors, according to a comparative example. (b) of FIG. 14 includes front and side images of a bag, which are captured by the CT device 1000 equipped with the scanner 100 composed of twenty modules 10 and twenty sources 12/detectors 15, a test example of the present invention. In (a) of FIG. 14, the shape of the bag and the shapes of the inside items in the side image are unclear. On the other hand, in (b) of FIG. 14, the shape of the bag and the shapes of the inside items in both the front and side images may be clearly identified. The CT device 1000 of the present invention may configure the scanner 100 to include a plurality of sources 12/detectors 15 by connecting the modules 10, thereby improving the image resolution of the subject 400 with a simple structure and process.

As described above, according to the CT device 1000 of the present invention, a plurality of modules may be freely assembled depending on factors such as the purpose of use, scanning speed, image resolution, and subject size. Additionally, the positions of the sources 12/detectors 15 may be freely changed to minimize the effect of scatter noise between fan beams.

Furthermore, according to the present invention, the source 12 and detector 15 may be disposed in the gantry 11 to minimize external radiation leakage, and components such as the step portions 11-1a and 11-2a, the shielding members 17, and the pins P1/holes P2 may be further adopted to improve shielding efficiency between the modules 10.

In addition, according to the present invention, the modules 10 may be freely moved along the rails 70 to conveniently repair, maintain, or remove/replace a faulty module.

While the present invention has been particularly shown and described with reference to embodiments thereof, it will be understood by one of ordinary skill in the art that various changes in form and details may be made therein without departing from the scope of the present invention as defined by the following claims.

1. Modules for a computed tomography (CT) device, the modules comprising:
a gantry for providing an internal space through which a subject is transported, and having a first surface corresponding to an outer circumferential surface and a second surface corresponding to an inner circumferential surface;
a source disposed on the second surface of the gantry to generate and emit radiation toward the subject; and
a detector disposed opposite the source on the second surface of the gantry to detect the radiation transmitted through the subject.

2. The modules of claim 1, wherein the gantry has a polygonal or circular column shape with both ends open.

3. The modules of claim 1, wherein at least parts under the first surface of the gantry are connected to rails provided parallel to a transport direction of the subject.

4. The modules of claim 1, further comprising:
a second gantry disposed adjacent to the gantry to provide an internal space through which the subject is transported, and having a first surface corresponding to an outer circumferential surface and a second surface corresponding to an inner circumferential surface;
a second source disposed on the second surface of the second gantry to generate and emit radiation toward the subject; and
a second detector disposed opposite the second source on the second surface of the second gantry to detect the radiation transmitted through the subject.

5. The modules of claim 4, wherein the source of the gantry and the second source of the second gantry are offset by an angle greater than at least 20° around a central axis of the internal space on a plane perpendicular to a transport direction of the subject.

6. The modules of claim 1, wherein at least one step portion is formed at a side of the gantry.

7. The modules of claim 4, wherein at least one step portion is formed at a side of the gantry,
wherein at least one second step portion is formed at a side of the second gantry, and
wherein the step portion and the second step portion have complementary shapes so as to fit together.

8. The modules of claim 6, wherein a shielding member for shielding radiation leakage from the internal space is disposed on at least a partial surface of the step portion.

9. The modules of claim 7, wherein sides of the gantry and the second gantry are disposed adjacent to each other, and
wherein shielding members for shielding radiation leakage from the internal space are disposed on at least partial surfaces of the step portion and the second step portion to seal a gap between the step portion and the second step portion.

10. The modules of claim 4, wherein a pin is formed on at least a part of a side of the gantry in a direction parallel to a transport direction of the subject, and
wherein a hole is formed in at least a part of a side of the second gantry, corresponding to the pin, to accommodate the pin.

11. The modules of claim 1, wherein the detector has an arc shape to maintain an equal distance from the source to every part of the detector.

12. The modules of claim 1, wherein the detector comprises:
a collimator comprising a shieling part for shielding the radiation, and a slit for transmitting the radiation; and
cells for detecting the transmitted radiation.

13. The modules of claim 12, wherein, when a width of the gantry in a transport direction of the subject is denoted by w, a distance between the source and the cells is denoted by L, a distance between the source and the collimator is denoted by I, a distance between the collimator and the cells is denoted by d, and a width of the cells is denoted by s, the collimator of the detector is configured to satisfy:
$$( Inequality ) w / L \leq ( w - s / 2 ) / I [ where L \geq I + d ] .$$

14. A computed tomography (CT) device for capturing a tomographic image along a transport direction of a subject, the CT device comprising:
a front portion provided to allow entrance of the subject and to shield radiation;
a scanner for providing an internal space to allow transportation of the subject from the front portion and to scan the subject; and
a rear portion provided to allow exit of the subject after passing through the scanner and to shield radiation,
wherein the scanner is configured by connecting a plurality of modules to each other in a direction parallel to the transport direction of the subject, and wherein each module comprises:
a gantry for providing an internal space through which the subject is transported, and having a first surface corresponding to an outer circumferential surface and a second surface corresponding to an inner circumferential surface;
a source disposed on the second surface of the gantry to generate and emit radiation toward the subject; and
a detector disposed opposite the source on the second surface of the gantry to detect the radiation transmitted through the subject.

15. The CT device of claim 14, wherein at least parts under the first surface of each module are connected to rails provided parallel to the transport direction of the subject.

16. The CT device of claim 15, wherein a space created between a specific module and other modules by moving the specific module along an extension direction of the rails is provided as a maintenance space.

17. The CT device of claim 15, wherein a specific module is removed from the rails by disassembling rail connectors provided between the specific module and the rails.

18. The CT device of claim 14, wherein step portions are formed at sides of the pair of adjacent modules in complementary shapes so as to fit together.

19. The CT device of claim 18, wherein shielding members for shielding radiation leakage from the internal space are disposed on at least partial surfaces of the step portions to seal a gap between the step portions.

20. The CT device of claim 14, wherein the detector comprises:
a collimator comprising a shieling part for shielding the radiation, and a slit for transmitting the radiation; and
cells for detecting the transmitted radiation, and
wherein, when a distance between the cells of Nth and (N−1) th modules among the plurality of modules is denoted by w, a distance between the source and the cells of the Nth module is denoted by L, a distance between the source and the collimator of the Nth module is denoted by I, a distance between the collimator and the cells of the Nth module is denoted by d, and a width of the cells of the Nth module is denoted by s, the collimator of the detector of the Nth module is configured to satisfy:
$$\begin{matrix}( Inequality ) \\ \underline{w / L \leq ( w - s / 2 ) / I [ where L \geq I + d ]} .\end{matrix}$$
